(19) United States
(12) Patent Application Publication
(10) Pub. No.: US 2015/0352264 A1
(43) Pub. Date: Dec. 10, 2015

(54) POLYMERIC VASCULAR STENT AND IN VIVO VISUALIZATION THEREOF

(71) Applicant: Abbott Cardiovascular Systems Inc., Santa Clara, CA (US)

(72) Inventors: Julia C. Fox, San Carlos, CA (US); Joel Harrington, Redwood City, CA (US); Stephen Pacetti, San Jose, CA (US)

(21) Appl. No.: 14/298,144

(22) Filed: Jun. 6, 2014

(51) Int. Cl. A61L 31/14; A61L 31/06

(52) U.S. Cl. CPC A61L 31/14; A61L 31/06

(57) ABSTRACT

This invention is directed to vascular stents that can be visualized in vivo through the use of fluorescent imaging.

### FIELD OF THE INVENTION

This invention relates to polymeric vascular stents and their in vivo visualization.

### BACKGROUND OF THE INVENTION

The traditional means of visualizing vascular stents during percutaneous coronary intervention has been the use of x-ray imaging, which was facilitated by the fact that stents initially were made of metals, which were well-suited for this imaging method due to their high x-ray attenuation. The advent of polymeric scaffold stents, which polymers generally exhibit low x-ray attenuation, has, however, necessitated a search for alternate means of device visualization. This need for discovery brings with it a potential added benefit. The initial approach to visualization of polymeric stents has involved the incorporation of radiopaque markers in the stent scaffold by which the placement of the stent could be observed using standard x-ray imaging. The search for alternative imaging modalities, however, offers the possibility to visualize the entire scaffold and to at least reduce and perhaps eliminate the exposure of patients and medical personnel to potentially harmful x-rays.

Over the past several decades, fluorescence-based imaging techniques have been developed for in vitro and in vivo fluorescence imaging with near-infrared (NIR) light. Fluorescence-based imaging in the NIR spectral window is biologically safe, has good penetration depth in tissues, and has an outstanding signal-to-noise ratio due to minimum interference from tissue auto-fluorescence. The present invention extends the use of NIR fluorescent imaging to the in vivo visualization of vascular stents.

### SUMMARY OF THE INVENTION

Thus, in one aspect the present invention relates to a vascular stent, comprising:

a stent body comprising a biocompatible, biodegradable block copolymer having a structural block and a fluorescent block; or

a stent body comprising a biocompatible, biodegradable block copolymer having a block with a Tg higher than 37° C. and a block having a Tg lower than room temperature, wherein the block with the Tg lower than room temperature forms a discrete amorphous phase within a matrix formed by the block with the Tg higher than 37° C., wherein a fluorescent substance is dispersed in the amorphous phase; or
a stent body comprising a biocompatible, biodegradable semi-crystalline homopolymer having a Tg higher than 37° C., wherein a fluorescent substance is dispersed in the amorphous phase of the semi-crystalline polymer; or
a stent body comprising a blend of at least two biocompatible, biodegradable polymers wherein on of the polymers is a semi-crystalline homopolymer and the other polymer is a copolymer, the blend of the two polymers having a Tg higher than 37° C. and wherein a fluorescent substance is dispersed in an amorphous phase of the blend; or
a stent body comprising a biocompatible, biodegradable polymer wherein the stent body is coated with a layer comprising a biocompatible, biodegradable block copolymer having a structural block and a fluorescent block; or
a biocompatible, biodegradable block copolymer having a block with a Tg higher than 37° C. and a block having a Tg lower than room temperature, wherein the block with the Tg lower than room temperature forms a discrete amorphous phase within a crystalline or semi-crystalline matrix formed by the block with the Tg higher than 37° C., wherein a fluorescent substance is dispersed in the amorphous phase.

In an aspect of the invention, the the structural block is selected from the group consisting of poly(L-lactide), poly(D-lactide), poly(D,L-lactide), poly(meso-lactide), poly(lactide-co-glycolide), poly(gylcolide-co-caprolactone), poly(L-lactide-co-D,L-lactide), polyhydroxyalkanoates and polydioxanones.

In an aspect of this invention, the fluorescent block comprises a near infrared light emitting block.

In an aspect of this invention, the near infrared light emitting block comprises a polymer comprising a diol, citric acid and an α-amino acid.

In an aspect of this invention, the block with a Tg higher than 37° C. is selected from the groups consisting of poly(L-lactide), poly(D-lactide), poly(D,L-lactide), poly(meso-lactide), poly(lactide-co-glycolide), poly(L-lactide-co-D,L-lactide) and combinations thereof.

In an aspect of this invention, the block with a Tg lower than room temperature is selected from the group consisting of poly(caprolactone), poly(tirmethylene carbonate), polydioxanone, poly(4-hydroxy butyrate) (PHB), poly(butylene succinate) and combinations thereof.

In an aspect of this invention, the semi-crystalline homopolymer is selected from the group consisting of poly(L-lactide), poly(D-lactide) and poly(glycolide).

In an aspect of this invention, the copolymer is selected from the group consisting of poly(L-lactide-caprolactone), poly(D,L-lactide-co-caprolactone), poly(glycolide-co-caprolactone), poly(L-lactide-co-trimethylene carbonate), poly(D,L-lactide-co-trimethylene carbonate), poly(glycolide-co-trimethylene carbonate),poly(L-lactide-co-dioxanone), poly(D,L-lactide-co-dioxanone) and poly(glycolide-co-dioxanone).

In an aspect of this invention, the fluorescent substance is a near infrared light emitting substance.

In an aspect of this invention, the fluorescent block or fluorescent substance is both a near infrared excitation and a near infrared emitting substance.

In an aspect of this invention, the near infrared light emitting substance is selected from the group consisting of indocyanine green, octreotate, a phthalocyanine, a cyanine derivative, green fluorescent protein, porphyrinato-zinc(II), a porphyrin, a zinc protoporphyrin, an ethynyl-bridged oligo(porphyrinato-zinc(II), an oligo zinc porphyrin, a natural amino acid, L-cysteine, L-serine, L-lysine, histidine, glycine, glutamine, glutamic acid, asparagine and a non-natural α-amino acid.

In an aspect of this invention, the fluorescent substance comprises a biocompatible, biostable small molecule.

In an aspect of this invention, the biocompatible, biostable small molecule is selected from the group consisting of butylated hydroxytoluene, methyl paraben, ethyl paraben, propyl paraben, sodium benzoate, benzoic acid, benzyl alcohol, phenoxyethanol, gentisic acid, butylated hydroxyanisole, ethyl benzoate, methyl gallate, ethyl gallate, propyl gallate, benzyl benzoate, resveratrol and α-tocopherol.

In an aspect of this invention, the near infrared light emitting substance comprises a near infrared-II light emitting substance.

In an aspect of this invention, the near infrared-II light emitting substance is selected from the group consisting of single walled carbon nanotubes, carbon nano-dots, a fullerene, graphite and a nano-diamond.

In an aspect of this invention, a portion of the vasculature in which the vascular stent is positioned is irradiated with an x-ray source to obtain an image of the vasculature itself and procedural accessories not containing fluorescent imaging substances.

In an aspect of this invention, the fluorescent image of the vascular stent is spatially registered and superimposed in real time on the image obtained from the x-ray irradiation.

In an aspect of this invention, the fluorescence image is displayed in color over the x-ray image.

In an aspect of this invention, the stent body comprising a biocompatible, biodegradable block copolymer having a structural block and a fluorescent block further comprises a coating comprising a drug reservoir layer.

In an aspect of this invention, the stent body comprising a coating thereon, further comprises a drug dispersed in the coating.

In an aspect of this invention, the stent body comprising a coating thereon, further comprises a separate drug reservoir layer.

### DETAILED DESCRIPTION OF THE INVENTION

It is understood that use of the singular throughout this application including the claims includes the plural and vice versa unless expressly stated otherwise. That is, “a,” “an” and “the” are to be construed as referring to one or more of whatever the word modifies. A non-limiting example is the use of the phrase “a layer,” or “the layer,” which is understood to include one such layer, two such layers or even more such layers unless it is expressly stated or is unambiguously obvious from the context that such is not intended. Likewise without limitation, “a fluorescent substance” or “the fluorescent substance” refers to a single such substance or to a mixture of two or more fluorescent substances unless, again, it is expressly stated or absolutely obvious from the context that such is not intended. The converse is also true, that is, reference to an item in the plural includes the singular unless it is otherwise unambiguously clear from the context that only the plural is intended.

As used herein, words of approximation such as, without limitation, “about” “substantially,” “essentially” and “approximately” mean that the word or phrase modified by the term need not be exactly that which is written but may vary from that written description to some extent. The extent to which the description may vary will depend on how great a change can be instituted and have one of ordinary skill in the art recognize the modified version as still having the properties, characteristics and capabilities of the modified word or phrase. In general, but with the preceding discussion in mind, a numerical value herein that is modified by a word of approximation may vary from the stated value by ±15%.

As used herein, the use of “preferred,” “preferably,” or “more preferred,” and the like refers to preferences as they existed at the time of filing of the patent application.

As used herein, an “implantable medical device” refers to any type of appliance that is totally or partly introduced, surgically or medically, into a patient's body or by medical intervention into a natural orifice, and which is intended to remain there after completion of the procedure. The duration of implantation may be essentially permanent, i.e., intended to remain in place for the remaining lifespan of the patient; until the device biodegrades and naturally disappears; or until it is physically removed. Examples of implantable medical devices include, without limitation, implantable cardiac pacemakers and defibrillators; leads and electrodes for the preceding; implantable organ stimulators such as nerve, bladder, sphincter and diaphragm stimulators, cochlear implants; prostheses, vascular grafts, self-expandable stents, balloon-expandable stents, stent-grafts, grafts, artificial heart valves and cerebrospinal fluid shunts.

As used herein, “device body” refers to a fully formed implantable medical device with an outer surface to which no coating or layer of material different from that of which the device itself is fabricated has been applied. By “outer surface” is meant any surface however spatially oriented that is in contact with bodily tissue or fluids. A common example of a “device body” is a BMS, i.e., a bare metal stent, which, as the name implies, is a fully-formed usable stent that has not been coated with a layer of any material different from the metal of which it is made on any surface that is in contact with bodily tissue or fluids. Of course, device body refers not only to BMSs but to any uncoated device regardless of what it is made of, in particular with regard to this invention, to vascular stents fabricated from polymers.

Presently preferred implantable medical devices of this invention are stents. A stent refers generally to any device used to hold tissue in place in a patient's body. Particularly useful stents, however, are those used for the maintenance of patency of a vessel in a patient's body when the vessel is narrowed or closed due to diseases or disorders including, without limitation, tumors (in, for example, bile ducts, the esophagus, the trachea/bronchi, etc.), benign pancreatic disease, coronary artery disease, carotid artery disease and peripheral arterial disease such as atherosclerosis, restenosis and vulnerable plaque. Vulnerable plaque (VP) refers to a fatty build-up in an artery thought to be caused by atherosclerosis and inflammation. The VP is covered by a thin fibrous cap that can rupture leading to blood clot formation. A stent can be used to strengthen the wall of the vessel in the vicinity of the VP and act as a shield against such rupture. A stent can be used in, without limitation, the neuro, carotid, coronary, pulmonary, aorta, renal, biliary, iliac, femoral and popliteal, as well as other peripheral, vasculatures. A stent can be used for the treatment or prevention of disorders such as, without limitation, thrombosis, restenosis, hemorrhage, vascular dissection or perforation, vascular aneurysm, chronic total occlusion, claudication, anastomotic proliferation, bile duct obstruction and ureter obstruction.

In addition to the above uses, stents may also be employed for the localized delivery of therapeutic agents to specific treatment sites in a patient's body. In fact, therapeutic agent delivery may be the sole purpose of the stent or the stent may be primarily intended for another use such as those discussed above with drug delivery providing an ancillary benefit.

A stent used for patency maintenance is usually delivered to the target site in a compressed state and then expanded to fit the vessel into which it has been inserted. Once at a target location, a stent may be self-expandable or balloon expandable.

In particular, a stent of this invention is a vascular stent, which refers to a stent that is implanted in the circulatory system, that is, the system of arteries and veins that transport blood throughout the body and presently even more specifically to a stent used for the treatment of obstructive diseases of the arterial vasculature.

As used herein, “optional” means that the element modified by the term may, but is not required to, be present.

As use herein, a “coating” or any form of the verb “to coat” refers to one or more thin layers of material that are disposed over a stent body and that generally conform to the topology of the stent body over which it is disposed.

As use herein, a layer that is “disposed over” an indicated substrate, e.g., without limitation, over a device body or over another layer, refers to a thin, sheet-like coating of the material applied directly to essentially the entire exposed surface of the indicated substrate. By “exposed surface” is meant any surface regardless of its physical location with respect to the configuration of the device that, in use, would be in contact with bodily tissues or fluids. “Disposed over” may, however, also refer to the application of the thin layer of material to an intervening layer that has been applied to the substrate, wherein the material is applied in such a manner that, were the intervening layer not present, the material would cover substantially the entire exposed surface of the substrate.

As used herein, “drug reservoir layer” refers to a layer of polymer or blend of polymers that has dispersed within its three-dimensional structure one or more therapeutic agents. A polymeric drug reservoir layer is designed such that, by one mechanism or another, e.g., without limitation, by elution or as the result of biodegradation of the polymer, the therapeutic substance is released from the layer into the surrounding environment. The drug reservoir layer may also act as a rate-controlling layer. As used herein, “rate-controlling layer” refers to a polymer layer that controls the rate of release of therapeutic agents or drugs into the environment. While a separate layer applied to a stent body of this invention would constitute a drug reservoir layer, it is possible to include a drug in the same layer as the fluorescent substance, in which case that layer would also necessarily be a “drug reservoir layer” but is simply not referred to as such herein.

A drug refers to any substance that, when administered in a therapeutically effective amount to a patient suffering from a disease, has a therapeutic beneficial effect on the health and well-being of the patient. A therapeutic beneficial effect on the health and well-being of a patient includes, but it not limited to: (1) curing the disease; (2) slowing the progress of the disease; (3) causing the disease to retrogress; or, (4) alleviating one or more symptoms of the disease. As used herein, a therapeutic agent also includes any substance that when administered to a patient, known or suspected of being particularly susceptible to a disease, in a prophylactically effective amount, has a prophylactic beneficial effect on the health and well-being of the patient. A prophylactic beneficial effect on the health and well-being of a patient includes, but is not limited to: (1) preventing or delaying on-set of the disease in the first place; (2) maintaining a disease at a reduced level once such level has been achieved by a therapeutically effective amount of a substance, which may be the same as or different from the substance used in a prophylactically effective amount; or, (3) preventing or delaying recurrence of the disease after a course of treatment with a therapeutically effective amount of a substance, which may be the same as or different from the substance used in a prophylactically effective amount, has concluded.

As used herein, “treating” refers to the administration of a therapeutically effective amount of a therapeutic agent to a patient known or suspected to be afflicted with a vascular disease.

As used herein, a “patient” refers to any living organism that might benefit from the application of the implantable medical device and compositions of this invention. Preferable the patient is a mammal and most preferably at present the patient is a human being.

A “therapeutically effective amount” refers to that amount of a therapeutic agent that will have a beneficial effect, which may be curative or palliative, on the health and well-being of the patient with regard to the vascular disease with which the patient is known or suspected to be afflicted. A therapeutically effective amount may be administered as a single bolus, as intermittent bolus charges, as short, medium or long term sustained release formulations or as any combination of these. As used herein, short-term sustained release refers to the administration of a therapeutically effective amount of a therapeutic agent over a period from about several hours to about 3 days. Medium-term sustained release refers to administration of a therapeutically effective amount of a therapeutic agent over a period from about 3 day to about 14 days and long-term refers to the delivery of a therapeutically effective amount over any period in excess of about 14 days.

Drugs that may be suitable for inclusion in a drug reservoir layer or in a fluorescent substance containing layer or, if desired, in both layers of a vascular stent of this invention include, without limitation, antiproliferative agents, anti-inflammatory agents, antineoplastics and/or antimitotics, antiplatelet, anticoagulant, antifibrin, and antithrombin drugs, cytostatic or antiproliferative agents, antibiotics, antiallergic agents and antioxidants.

Suitable antiproliferative agents include, without limitation, actinomycin D, taxol, docetaxel, paclitaxel, sirolimus, everolimus, zotarolimus, umirolimus, biolimus, deforolimus, temsirolimus, merilimus, myolimus, novolimus, FKBP-12 mediated mTOR inhibitors, perfenidone and prodrugs, co-drugs and combinations thereof.

Suitable anti-inflammatory agents include, without limitation, clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus and prodrugs, co-drugs and combinations thereof.

Suitable antineoplastics and/or antimitotics include, without limitation, paclitaxel, docetaxel, methotrexate, azathioprine, vincristine, vinblastine, fluorouracil, doxorubicin hydrochloride, and mitomycin.

Suitable antiplatelet, anticoagulant, antifibrin, and antithrombin drugs include, without limitation, sodium heparin, low molecular weight heparins, heparinoids, hirudin, argatroban, forskolin, vapiprost, prostacyclin, prostacyclin dextran, D-phe-pro-arg-chloromethylketone, dipyridamole, glycoprotein IIb/IIIa platelet membrane receptor antagonist antibody, recombinant hirudin and thrombin, thrombin inhibitors such as Angiomax a, calcium channel blockers (such as nifedipine), colchicine, fish oil (omega 3-fatty acid), histamine antagonists, lovastatin, monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol and combinations thereof.

Suitable cytostatic (antiproliferative) agents include, without limitation, angiopeptin, angiotensin converting enzyme inhibitors such as captopril, cilazapril or lisinopril, calcium channel blockers such as nifedipine; colchicine, fibroblast growth factor (FGF) antagonists; fish oil (ω-3-fatty acid); histamine antagonists; lovastatin, monoclonal antibodies such as, without limitation, those specific for Platelet-Derived Growth Factor (PDGF) receptors; nitroprusside, phosphodiesterase inhibitors, prostaglandin inhibitors, suramin, serotonin blockers, steroids, thioprotease inhibitors, triazolopyrimidine (a PDGF antagonist) and nitric oxide.

Other suitable therapeutic agents include, without limitation, alpha-interferon, genetically engineered epithelial cells, DNA and RNA nucleic acid sequences, antisense molecules, ribozymes, antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides; antiviral agents; analgesics; anorexics; antihelmintics; antiarthritics, antiasthmatic agents; anticonvulsants; antidepressants; antidiuretic agents; antidiarrheals; antihistamines; antimigrain preparations; antinauseants; antiparkinsonism drugs; antipruritics; antipsychotics; antipyretics; antispasmodics; anticholinergics; sympathomimetics; xanthine derivatives; cardiovascular preparations including calcium channel blockers, beta-blockers such as pindolol, antiarrhythmics; antihypertensives; diuretics; vasodilators including general coronary; peripheral and cerebral; central nervous system stimulants; hypnotics; immunosuppressives; muscle relaxants; parasympatholytics; psychostimulants; sedatives; tranquilizers and natural or genetically engineered lipoproteins.

As used herein, “biocompatible” refers a substance such as a polymer, an organic small molecule or an inorganic small molecule that in its intact form and in the form(s) it assumes during decomposition or degradation are not harmful to a living organism.

As used herein, “biodegradable” refers to any natural means by which a polymer can be disposed of in a patient's body. This includes such phenomena as, without limitation, biological decomposition, bioerosion, absorption, resorption, etc. Biodegradation of a polymer in vivo results from the action of endogenous biological agents and conditions such as, without limitation, enzymes, microbes, cellular components, physiological pH, hydrolysis, reactive oxygen species, free radicals, temperature and the like. Bioabsorbable or bioresorbable on the other hand generally refers to the situation wherein the polymer itself or its degradation products are removed from the body by cellular activity such as, without limitation, phagocytosis. Bioerodible refers to both physical processes such as, without limitation, dissolution and chemical processes such as, without limitation, backbone cleavage by hydrolysis of the bonds linking constitutional units of a polymer together. As used herein, biodegradable

As used herein, a polymer refers to a macromolecule formed of repeating subunits known as monomers or constitutional units, wherein a “constitutional unit” refers to the monomer as it appears when incorporated into a polymer. For example, lactic acid, CH₃CH(OH)C(O)OH is a monomer and —OCH(CH₃)C(O)— is the constitutional unit provided by lactic acid. Generally polymers have a relatively high molecular weight, often into the range of millions of grams/mol, but for the purposes of this invention a compound with two or more repeats of each constitutional unit will be considered a polymer. A polymer of this invention may be a homopolymer or a copolymer, which may in turn be an alternating, random or block copolymer.

A homopolymer refers to a polymer comprising a single monomer, a monomer simply being a molecule that is iteratively reacted with itself to form chains of constitutional units, i.e., to form a homopolymer. A copolymer refers to a polymer prepared from two or more monomers that may be reacted so as to form a random copolymer, a regular alternating copolymer, a block copolymer or a star copolymer. A random copolymer has the general structure, assuming two monomers/constitutional units, x-x-y-x-y-y-y-x-y-y-x-x-x- . . . , while a regular alternating copolymer has the general structure: . . . x-y-x-y-x-y- . . . , it being understood that the juxtaposition of constitutional units shown is for purpose of illustration only and a copolymer of this invention may vary from that shown. A block copolymer has the general structure: . . . x-x-x-y-y-y-x-x-x-y-y-y- . . . Similarly to random and alternating copolymers, the number of constitutional units in each block and the number of blocks in a block copolymer of this invention are not in any manner limited by the preceding illustrative generic structure. For instance, a “fluorescent block” of a block copolymer of this invention can comprise three constitution units, a polyol, an α-amino acid and citric acid.

A semi-crystalline homopolymer refers to a homopolymer that consists of both crystalline regions in which portions of the polymer molecules arrange, internally, with other molecules or both, in ordered structures and amorphous regions where the polymer molecules or portions thereof are randomly disposed.

As used herein, a “polyol” refers to an alcohol containing at least two hydroxyl groups. Presently preferred are α,ω-dihydroxy alcohols such as 1,4-butanediol, 1,8-octanediol and 1,12-dodecanediol.

As used herein, an “α-amino acid” refers to a carboxylic acid having a free amino group bonded to the a carbon atom. An α-amino acid of this invention may be a natural amino acid, as such are known and referred to into the chemical arts, a derivative of such natural α-amino acid or it may be a purely synthetic α-amino acid.

A block copolymer may be further designated as a diblock, triblock, tetrablock or, for any polymer consisting of more than two blocks, simply a “multi-block” copolymer, which, as should be apparent, simply refers to the number of discrete blocks identifiable in the polymer.

In some embodiments of this invention the glass transition temperature, Tg, of a block is specified in order to achieve the desired result. The Tg is a second order thermodynamic transition and is the reversible transition of an amorphous polymer or the amorphous regions within a semi-crystalline polymer from a hard, glassy, relatively brittle state to a molten or rubbery state. The phenomenon is extremely well-known to those skilled in the polymer arts and needs no further explanation here. For the purposes of this invention a biocompatible, biodegradable block copolymer that does not include a fluorescent block as a segment of the copolymer per se requires a block with a Tg higher than 37° C., which is essentially body temperature of a human. This block, then, will maintain the strength, toughness, tensile modulus and relative rigidity of the block copolymer under normal room temperature conditions, which, for the purposes of this invention is considered to be between about 15° C. and about 25° C. and under the temperature conditions found in the mammalian, in particular human, body. On the other hand, the block of the block copolymer that has a Tg less than room temperature will maintain an amorphous, rubbery state under all temperature operating conditions likely to be experienced by a vascular stent of this invention. In this manner, when a fluorescent substance is blended with the block copolymer it will tend to migrate to and locate in the discrete, amorphous low Tg phase of the block copolymer where it will encounter significantly more freedom of molecular movement so as to be able to attain a conformationally lowest energy state for maximum fluorescence.

The same result, that is, the localization of a fluorescent substance in an amorphous region of a polymer where it is free to achieve a conformationally lowest energy state can also be achieved with a semi-crystalline homopolymer. That is, in manufacturing processes where the polymer crystalline phase is disassembled or melted as by polymer dissolution or melt extrusion, during the process of recrystallization an added fluorescent substance will be excluded from the crystalline phase and localized in the amorphous phase. Homopolymers that participate in this phenomenon include, without limitation poly(L-lactide), poly(D-lactide) and poly(glycolide).

Similarly, such localization of a fluorescent substance can include a blend of polymers wherein one or more of the polymers comprise a semi-crystalline homopolymer as above and the other polymers of the blend comprise copolymers wherein the Tg of the entire blend is higher than 37° C. The blend should form an amorphous phase in which an added fluorescent substance will disperse.

As used herein, a “structural block” of a biocompatible, biodegradable block copolymer refers to a block that is selected to confer desired physical properties such as strength, toughness, tensile modulus, stiffness, etc., to the copolymer. Polymers and copolymers providing such properties are well-known to those skilled in the art. Among the polymeric substances from which the structural block may be selected are, without limitation, poly(L-lactide) (PLLA), poly(D-lactide) (PDLA), polymandelide (PM), polyglycolide (PGA), poly(L-lactide-co-D,L-lactide) (PLDLA), poly(D,L-lactide) (PDLLA), 96/4 poly(D,L-lactide) (PDLLA), poly(D,L-lactide-co-glycolide) (PLGA), poly(L-lactide-co-caprolactone), and poly(L-lactide-co-glycolide) (PLLGA). The poly(L-lactide-co-caprolactone) may have 1 to 5% (by mole or weight) of caprolactone. With respect to PLLGA, the stent scaffolding can be made from PLLGA with a mole % of GA between 5-15 mol %. The PLLGA can have a mole % of (LA:GA) of 85:15 (or a range of 82:18 to 88:12), 95:5 (or a range of 93:7 to 97:3), or commercially available PLLGA products identified as being 85:15 or 95:5 PLLGA. Polymers that are more flexible or that have a lower modulus than those mentioned above may also be used. Exemplary lower modulus bioabsorbable polymers include, polycaprolactone (PCL), poly(trimethylene carbonate) (PTMC), polydioxanone (PDO), poly(4-hydroxy butyrate) (PHB), and poly(butylene succinate) (PBS), and blends and copolymers thereof. These examples are not the only polymers that may be used as the structural block. Many other examples can be provided, such as those found in Polymeric Biomaterials, second edition, edited by Severian Dumitriu; chapter 4.

As used here, a “fluorescent block” of a biocompatible, biodegradable block copolymer refers to a block that, obviously, fluoresces when exposed to a certain wavelength of light. Of particular note are those polymers that absorb light in the visible to infrared region of the electromagnetic spectrum, that is 500-700 nm and fluoresce in the NIR region, 700-1300 nm, preferably at present 700-900 nm. Fluorescence-based imaging in this window is biologically safe, has good tissue penetration depth and exhibits excellent signal-to-noise ratios due to minimal interference from tissue auto-fluorescence. In addition, fluorescence-based optical imaging exhibits high temporal and spatial resolution and fast acquisition times, which permits precise quantification of vascular anatomy and hemodynamics.

Of course, the excitation source must have sufficient energy density at the tissue depth to give a fluorescence signal. Strategies to maximize the signal to noise ratio (SNR) include strong absorbers coupled with NIR detection often gated for “long” (microseconds) fluorescence lifetimes from large molecules.

The primary enabling technologies forfluorescence-based imaging have been expanding availability of biologically compatible near-infrared probes, development of highly sensitive photon-detection technologies and advanced data processing techniques.

As used herein, a “fluorescent substance” refers to (1) a substance that absorbs in the infrared region of the electromagnetic spectrum, is “excited,” that is, raised to a higher energy level and then fluoresces, release that energy, in the NIR region. Examples of such substances include, without limitation, indocyanine green, octreotate, a phthalocyanine, a cyanine derivative, green fluorescent protein, porphyrinato-zinc(II), a porphyrin, a zinc protoporphyrin, an ethynyl-bridged oligo(porphyrinato-zinc(II), an oligo zinc porphyrin, natural amino acid, in particular L-cysteine, L-serine, L-lysine, histidine, glycine, glutamine, glutamic acid, asparagine and non-natural α-amino acids; (2) a biocompatible, biostable small molecule that fluoresces, examples of which include, without limitation, butylated hydroxytoluene, methyl paraben, ethyl paraben, propyl paraben, sodium benzoate, benzoic acid, benzyl alcohol, phenoxyethanol, gentisic acid, butylated hydroxyanisole, ethyl benzoate, methyl gallate, ethyl gallate, propyl gallate, benzyl benzoate, resveratrol and α-tocopherol; and (3) NIR-II light emitting substances, which includes, without limitation, single walled carbon nanotubes, carbon nano-dots, fullerenes, graphite and nano-diamonds.

The focus this invention is primarily directed to the emission and detection of NIR wavelength electromagnetic energy due to the so-called NIR window of transparency, that is a region of the electromagnetic spectrum to which mammalian tissues are essentially transparent. This permits the emitted energy to traverse substantially greater distances through tissue and still be intense enough to enable ready detection. the NIR spectrum is approximately 650 nm to 1.4 μm. From this range, two sub-ranges have been identified, NIR-I, approximately 750 nm to 900 nm and NIR-II, approximately 1.1 μm to 1.4 μm. So long as the emission wavelength of a fluorescence substance is in the range, that substance is within the scope of this invention. It is presently preferred, however, that both excitation wavelength and emission wavelength be within the NIR spectrum. In this manner, both excitation and emission take advantage of the NIR window of transparency such that fluorescent substances in deeper tissues may be readily excited and their emissions readily detected. Examples, without limitation, of fluorescent substances that possess this characteristic are (Cy-7 from Lumiprobe Corporation, Hallandale Beach, Fla.), which excites at 750 nm and emits at 773 nm and (Cy-7.5, likewise from Lumiprobe), which excites at 788 nm and emits at 808 nm. Other such fluorescent substances will be apparent to those skilled in the art based on the disclosure herein.

Alternatives technologies that are considered feasible in the context of this invention are confocal or multiphoton excitation microscopy, due to the ability to use smaller molecules for conjugation of the chromophore. Using two-photon excitation allows both excitation and the detection within the NIR window. This removes the need for high intensities, drastically reduces the scatter and can help the SIN especially if coupled with confocal imaging of the fluorescence. More importantly, a two-photon excitation at 800-1300 nm is energetically equivalent to single photon absorption at 400-650 nm near the focus of the incoming light. More chromophores are possible in this range with fewer conjugated bonds. Light sources then need to be short pulse lasers (pico, femtosecond range) used below the damage thresholds of the tissue.

Without adding a chromophore, autofluorescence of arteries has been demonstrated with fast acquisition times and low SNR. It is possible, then, that a scaffold of poly(L-lactide), for example, might be visible during implantation without an added chromophore. This would require fast electronics and signal processing.

Advantages offluorescence-based optical imaging over x-ray imaging include no radiation dose and no risk of nephrotoxicity due to elimination of the need for contrast dye. In addition to visualizing the location and deployment of the scaffold within the blood vessel, NIR imaging could be used to characterize in vivo the degree of stenosis of the vessels. Because of its temporal resolution, NIR may also be useful for imaging changes in vessel diameter due to active vasodilation or vasoconstriction. This would be particularly interesting for visualizing the long-term benefit of polymeric absorbable scaffolds, which degrade over time and allow the return of native vessel motion.

If, however, it is desired that the vasculature itself be visualized, it is possible to include x-ray imaging but at substantially lower time-of-exposures and therefore total dosage to achieve such imaging. The fluorescent image of the vascular stent could then be spatially registered and superimposed in real time on the image obtained from the x-ray irradiation. To increase the contrast between the images, the fluorescent image of the vascular stent could be displayed in color. Using this dual mode of visualization, “procedural accessories” such as, but not limited to, catheters, guidewires and the like could also be visualized to provide an even more complete image of the treatment site.

While embodiments of the present invention have been discussed with varying degrees of specificity, it is understood that the disclosures herein are not intended, nor should they be interpreted, to limit the scope of this invention and those skilled in the art will be able to visualize changes and extensions of the concepts set forth herein; such changes and extensions are within the aegis of this invention.

What is claimed:

1. A vascular stent, comprising:
a stent body comprising a biocompatible, biodegradable block copolymer having a structural block and a fluorescent block; or
a stent body comprising a biocompatible, biodegradable block copolymer having a block with a Tg higher than 37° C. and a block having a Tg lower than room temperature, wherein the block with the Tg lower than room temperature forms a discrete amorphous phase within a matrix formed by the block with the Tg higher than 37° C., wherein a fluorescent substance is dispersed in the amorphous phase; or
a stent body comprising a biocompatible, biodegradable semi-crystalline homopolymer having a Tg higher than 37° C., wherein a fluorescent substance is dispersed in the amorphous phase of the semi-crystalline polymer; or
a stent body comprising a blend of at least two biocompatible, biodegradable polymers wherein on of the polymers is a semi-crystalline homopolymer and the other polymer is a copolymer, the blend of the two polymers having a Tg higher than 37° C. and wherein a fluorescent substance is dispersed in an amorphous phase of the blend; or
a stent body comprising a biocompatible, biodegradable polymer wherein the stent body is coated with a layer comprising a biocompatible, biodegradable block copolymer having a structural block and a fluorescent block; or
a biocompatible, biodegradable block copolymer having a block with a Tg higher than 37° C. and a block having a Tg lower than room temperature, wherein the block with the Tg lower than room temperature forms a discrete amorphous phase within a crystalline or semi-crystalline matrix formed by the block with the Tg higher than 37° C., wherein a fluorescent substance is dispersed in the amorphous phase.

2. The vascular stent of claim 1, wherein the structural block is selected from the group consisting of poly(L-lactide), poly(D-lactide), poly(D,L-lactide), poly(meso-lactide), poly(lactide-co-glycolide), poly(gylcolide-co-caprolactone), poly(L-lactide-co-D,L-lactide), polyhydroxyalkanoates and polydioxanones

3. The vascular stent of claim 1, wherein the fluorescent block comprises a near infrared light emitting block.

4. The vascular stent of claim 3, wherein the near infrared light emitting block is also excited by near infrared light.

5. The vascular stent of claim 1, wherein the near infrared light emitting block comprises a polymer comprising a diol, citric acid and an α-amino acid.

6. The vascular stent of claim 1, wherein the block with a Tg higher than 37° C. is selected from the groups consisting of poly(L-lactide), poly(D-lactide), poly(D,L-lactide), poly(meso-lactide), poly(lactide-co-glycolide), poly(L-lactide-co-D,L-lactide) and combinations thereof.

7. The vascular stent of claim 1, wherein the block with a Tg lower than room temperature is selected from the group consisting of poly(caprolactone), poly(tirmethylene carbonate), polydioxanone, poly(4-hydroxy butyrate) (PHB), poly(butylene succinate)and combinations thereof.

8. The vascular stent of claim 1, wherein the semi-crystalline homopolymer is selected from the group consisting of poly(L-lactide), poly(D-lactide) and poly(glycolide).

9. The vascular stent of claim 1, wherein the copolymer is selected from the group consisting of poly(L-lactide-caprolactone), poly(D,L-lactide-co-caprolactone), poly(glycolide-co-caprolactone), poly(L-lactide-co-trimethylene carbonate), poly(D,L-lactide-co-trimethylene carbonate), poly(glycolide-co-trimethylene carbonate),poly(L-lactide-co-dioxanone), poly(D,L-lactide-co-dioxanone) and poly(glycolide-co-dioxanone).

10. The vascular stent of claim 1, wherein the fluorescent substance is a near infrared light emitting substance.

11. The vascular stent of claim 10, wherein the near infrared light emitting substance is also excited by a near infrared light.

12. The vascular stent of claim 7, wherein the near infrared light emitting substance is selected from the group consisting of indocyanine green, octreotate, a phthalocyanine, a cyanine derivative, green fluorescent protein, porphyrinato-zinc(II), a porphyrin, a zinc protoporphyrin, an ethynyl-bridged oligo(porphyrinato-zinc(II), an oligo zinc porphyrin, a natural amino acid, L-cysteine, L-serine, L-lysine, histidine, glycine, glutamine, glutamic acid, asparagine and a non-natural α-amino acid.

13. The vascular stent of claim 1, wherein the fluorescent substance comprises a biocompatible, biostable small molecule.

14. The vascular stent of claim 1, wherein the biocompatible, biostable small molecule is selected from the group consisting of butylated hydroxytoluene, methyl paraben, ethyl paraben, propyl paraben, sodium benzoate, benzoic acid, benzyl alcohol, phenoxyethanol, gentisic acid, butylated hydroxyanisole, ethyl benzoate, methyl gallate, ethyl gallate, propyl gallate, benzyl benzoate, resveratrol and α-tocopherol.

15. The vascular stent of claim 1, wherein the near infrared light emitting substance comprises a near infrared-II light emitting substance.

16. The vascular stent of claim 15, wherein the near infrared-II light emitting substance is selected from the group consisting of single walled carbon nanotubes, carbon nano-dots, a fullerene, graphite and a nano-diamond.

17. The vascular stent of claim 1, wherein a portion of the vasculature in which the vascular stent is positioned is irradiated with an x-ray source to obtain an image of the vasculature itself and procedural accessories not containing fluorescent imaging substances.

18. The vascular stent of claim 17, wherein the fluorescent image of the vascular stent is spatially registered and superimposed in real time on the image obtained from the x-ray irradiation.

19. The vascular stent of claim 18, wherein the fluorescence image is displayed in color over the x-ray image.

20. The vascular stent of claim 1, wherein the stent body comprising a biocompatible, biodegradable block copolymer having a structural block and a fluorescent block further comprises a coating comprising a drug reservoir layer.

21. The vascular stent of claim 1, wherein the stent body comprising a coating thereon, further comprises a drug dispersed in the coating.

22. The vascular stent of claim 1, wherein the stent body comprising a coating thereon, further comprises a separate drug reservoir layer.
